(19) United States
(12) Patent Application Publication
(10) Pub. No.: US 2025/0209042 A1
(43) Pub. Date: Jun. 26, 2025

(54) SEQUENCE DATA PROCESSING, RETENTION, AND RECOVERY

(71) Applicant: Illumina, Inc., San Diego, CA (US)

(72) Inventors: Paul SMITH, San Diego, CA (US); Guillaume RIZK, Rennes (FR); Michael CARNEY, San Diego, CA (US)

(73) Assignee: Illumina, Inc., San Diego, CA (US)

(21) Appl. No.: 18/987,285

(22) Filed: Dec. 19, 2024

Related U.S. Application Data
(60) Provisional application No. 63/613,287, filed on Dec. 21, 2023.

(51) Int. Cl. G06F 16/174; G16B 30/00; G16B 50/50

(52) U.S. Cl. CPC G06F 16/1744; G16B 30/00; G16B 50/50

(57) ABSTRACT

A sequence data processing and retention method includes obtaining sequence data produced by a sequencer device. The sequence data includes genomic data of interest and metadata. The method processes the sequence data, and this processing includes separating the genomic data of interest from the metadata, and compressing the separated genomic data of interest based on a reference sequence to produce compressed genomic data The method additionally stores storing the compressed genomic data and the metadata. Optionally, based on a request, a process recovers the sequence data from the stored compressed genomic data and metadata, where the recovering includes decompressing the compressed genomic data to provide decompressed genomic data of interest as the separated genomic data of interest, and combining the decompressed genomic data of interest with the metadata to provide combined genomic data and metadata.

### BACKGROUND

Genomic sequencing describes a method of identifying nucleotides or other component parts of genomic data. A nucleic acid sequencing device, also referred to as a sequencer, generates data as base calls, for instance ones corresponding to, or representing, nucleotides of a ribonucleic acid (RNA) or deoxyribonucleic acid (DNA) fragment sequenced by the nucleic acid sequencing device. A read sequence includes data that corresponds to a series of these nucleotide base calls as well as data describing quality scores for the series of nucleotides. This data is usually output from the sequencing device as a plurality of records (‘sequence’ or ‘sequencing’ data) for analysis/processing by a computer system, for instance to correlate component parts, such as nucleotides, with respective positions in a given reference genome.

A well-known format for outputting sequence data is the Binary Base Call format, known as “BCL” format. The BCL format is not commonly used directly in secondary analysis. For instance, the BCL data is often demultiplexed prior to performing further processing. BCL data is relatively large in size. To the extent that it is provided by the sequencing device to other device(s) for processing, these relatively large BCL data file(s) then must be managed, stored, and processed by downstream devices. In addition, BCL files do not compress particularly well—the well-known GZIP compression algorithm is effective to reduce the file size by only about 30% in many cases. There are other drawbacks in some implementation-specific situations. For instance, in some flow cell-based sequencing applications, the first approximately 25 cycles produce data for all nanowells, increasing the size of the resultant BCL files, requiring additional filter files, and creating later work to remove empty wells.

### SUMMARY

Various approaches have been developed for storing sequence/sample data in varying formats and processed through varying compression algorithms. The FASTQ format is effectively an open standard for storing sample data in a human readable (text-based) format. Some compression techniques in the genomic sequencing space are reference-free, in which the compression is based on similarity between the read data, for example. Other techniques are reference-based, in which the compression is based on similarity between the read data and a reference sequence. A common approach to storing sequence data, for instance BCL data, produced by the sequencer is to convert the sequence data into the FASTQ format in FASTQ file(s), compress (‘zip’) the FASTQ file(s) using the GZIP algorithm as one example, and then store the compressed reformatted data.

In creating per-sample FASTQ data, information that appears in the original sequencing data is lost. This can be problematic if a mistake is made in the conversion of the sequence data to the per-sample data. The ORA compression format is based on FASTQ and is another (lossy) format in which sequence data may be compressed and stored. ORA is more efficient but suffers from some of the same limitations as FASTQ. For instance, a sample can be undefined if the sample sheet, for instance a configuration file or other specification that maps indexes to samples, is missing or corrupt, the indexes are entered incorrectly, or a mistake is made in the demultiplexing operation. Moreover, the data could be trimmed incorrectly if the sample sheet is missing.

As a result of these and other drawbacks, and although cumbersome to maintain the original sequence data files, e.g., BCL files, in practice it is common to maintain the raw sequencing data files from the sequencer so that all of the original information (including adapter and index information, for instance) remains available in case it was needed.

Shortcomings of the prior art are overcome and additional advantages are provided through the provision of a computer-implemented method. The method obtains sequence data produced by a sequencer device, the sequence data including genomic data of interest and metadata. The method also processes the sequence data, the processing including: separating the genomic data of interest from the metadata; and compressing the separated genomic data of interest based on a reference sequence to produce compressed genomic data. The method further stores the compressed genomic data and the metadata.

Another example computer-implemented method includes, based on a request to recover sequence data from stored compressed genomic data and metadata, obtaining the stored compressed genomic data and the metadata, the compressed genomic data including genomic data of interest compressed based on a reference sequence, decompressing the compressed genomic data to provide decompressed genomic data of interest, and combining the decompressed genomic data of interest with the metadata to provide the sequence data.

Yet another example computer-implemented method includes obtaining sequence data produced by a sequencer device, the sequence data including genomic data of interest and metadata, processes the sequence data, the processing including compressing the genomic data of interest and metadata based on a reference sequence to produce compressed data, and storing the compressed data.

A further example computer-implemented method includes, based on a request to recover sequence data from stored compressed genomic data and metadata, obtaining the compressed genomic data and metadata, the compressed genomic data and metadata including genomic data of interest compressed based on a reference sequence, and decompressing the compressed genomic data and metadata to provide decompressed genomic data of interest and metadata as the sequence data.

Additional aspects of the present disclosure are directed to systems and computer program products configured to perform the methods described above and herein. The present summary is not intended to illustrate each aspect of, every implementation of, and/or every embodiment of the present disclosure. Additional features and advantages are realized through the concepts described herein.

### BRIEF DESCRIPTION OF THE DRAWINGS

Aspects described herein are particularly pointed out and distinctly claimed as examples in the claims at the conclusion of the specification. The foregoing and other objects, features, and advantages of the disclosure are apparent from the following detailed description taken in conjunction with the accompanying drawings in which:

FIG. 1 depicts an example conventional workflow for sequence data formatting and compression;

FIG. 2 depicts a conceptual representation of a demultiplexing operation to provide per-sample data;

FIG. 3 depicts a conceptual representation of a transpose operation;

FIG. 4 depicts a conceptual representation of a trim operation;

FIG. 5 depicts another example conventional workflow for sequence data formatting and compression;

FIG. 6 depicts an example workflow for sequence data formatting and compression in accordance with aspects described herein;

FIG. 7 depicts an example workflow for on-instrument sequence data formatting and compression in accordance with aspects described herein;

FIG. 8 depicts an example of a recovery scenario, in accordance with aspects described herein;

FIG. 9 depicts an example process for sequence data processing and retention, in accordance with aspects described herein;

FIG. 10 depicts an example process for sequence data recovery, in accordance with aspects described herein; and

FIG. 11 depicts one example of a computer system and associated devices to incorporate and/or use aspects described herein.

### DETAILED DESCRIPTION

Described herein are approaches for sequence data processing, retention, and recovery. Aspects are presented by way of example, and not limitation, in the context of extending the ORA compression format. Like the ORA format, aspects described herein can provide data that is per-sample and compressed using a reference sequence, i.e., by way of reference-based compression. Sample and/or reference sequences could be genomic sequences of human or non-human organisms, and therefore aspects described herein can compress genomic data sampled from human or non-human organisms and/or using reference sequences of human or non-human organisms. Although aspects described herein may make reference to human genomic sequences, the techniques described can be applied to various non-human genomic samples or references, e.g., Sus scrofa (pig), Gallus gallus (chicken), Oryza sativa (Japanese rice), Arabidopsis thaliana, Triticum aestivum (bread wheat), Bos taurus (cattle), Glycine max (soybean), Rattus norvegicus (norway rat), Zea mays (maize), Danio rerio (zebrafish), Mus musculus (house mouse), Caenorhabditis elegans (roundworm), and others, as examples.

Aspects described herein differ from conventional ORA format in some ways. For instance, all of the bases used for indexing and adapter information that are represented in, and read from, the raw sequencing data file(s) are maintained, and the data is soft trimmed so that the adapter information and index information are maintained. Further, the data can be restored, re-demultiplexed and re-trimmed to reproduce the per-sample genomic data in the event of an error. Additionally, the location of each cluster in the sequence data could optionally be discarded. Conventionally this x-y coordinate data of each cluster was maintained for demultiplexing, transposing, trimming, and/or compressing operations and included in the compressed FASTQ data and subsequent ORA data produced therefrom, despite not being useful to that processing.

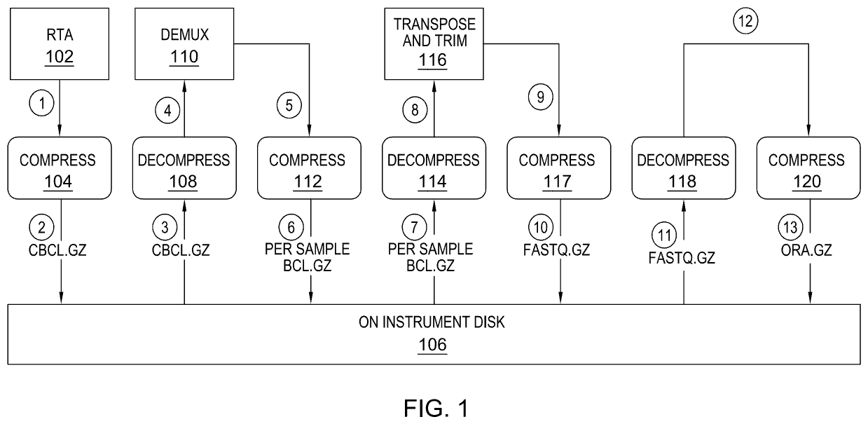

FIG. 1 depicts an example conventional workflow for sequence data formatting and compression. Primary sequencing data analysis software 102, for instance the Real-Time Analysis (RTA) software offered by Illumina Inc., produces sequence data (1) containing information for multiple samples, for instance genomic data of interest and metadata. One or more processes can process this data. For instance, the data is compressed (104) as zipped concatenated BCL (CBCL.gz) file(s) in this example, and stored (2) to a storage device 106. In this example, the storage device is an on-instrument storage disk of the sequencing instrument. In specific examples, the sequencing data is accumulated in working memory of the instrument during cycles of sequencing chemistry and imaging, providing base calls and associated quality scores representing the primary structure of DNA or RNA strands, and written to a storage device, such as a ‘hard drive’, solid-state disk/drive, or other non-volatile storage of the instrument.

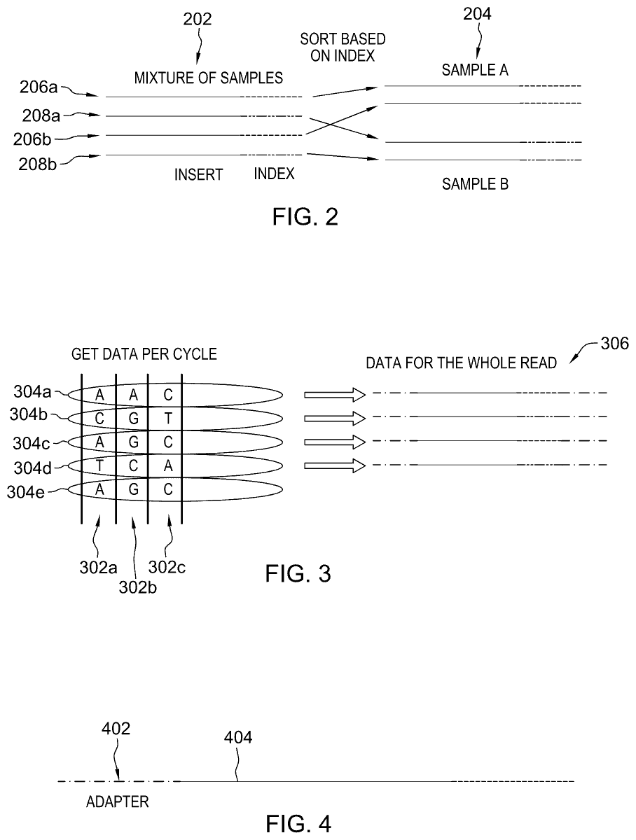

The workflow continues by reading (3) each CBCL.gz file from disk 106 into working memory, uncompressing/unzipping (108) the CBCL.gz data, and providing (4) this for a demultiplexing (demux) operation 110 on the uncompressed CBCL data. In the example of FIG. 1, the demultiplexing operation results in per-sample BCL data (5). FIG. 2 depicts a conceptual representation of a demultiplexing operation to provide per-sample data. With reference to FIG. 2, the input data 202 to the demultiplexing operation is a mixture of fragmented/portioned sample data tagged with indexes to identify which fragments correspond to which samples. In this example, all sample data concatenated with a red index (i.e., 206a, 206b) corresponds to a first sample (Sample A), and all sample data concatenated with a cyan index (i.e., 208a, 208b) corresponds to a second sample (Sample B). The demultiplexing operation sorts the input data 202 based on index to provide per-sample data 204 (two samples A and B are depicted in this example).

Returning to FIG. 1, the workflow compresses/zips (112) the per-sample data output from the demultiplexing operation (110) into compressed per-sample BCL data (BCL.gz) and stores (6) this to disk 106. One reason to retain the raw sequencing data (e.g., BCL data) in this conventional approach is that if a mistake was made in the demultiplexing operations, for instance there was a mistake in the sample sheet, the raw data was needed to address that and properly re-demultiplex the raw data.

The workflow continues by reading (7) from disk (106) and decompressing (114) the compressed per-sample BCL data for input (8) to transpose and trim operations 116. The resulting data is compressed (117) and stored (10) back to disk 106 as compressed FASTQ data (FASTQ.GZ). Optionally, in alternative scenarios, compression 112 and decompression 114 can be skipped/omitted. In these situations, the output of demux 110 can be maintained in memory, and this data can feed the transpose/trim operation 116 directly. In this case, the first per-sample information on disk would be the FASTQ data is written after compression at 117. FIG. 3 depicts a conceptual representation of a transpose operation and FIG. 4 depicts a conceptual representation of a trim operation.

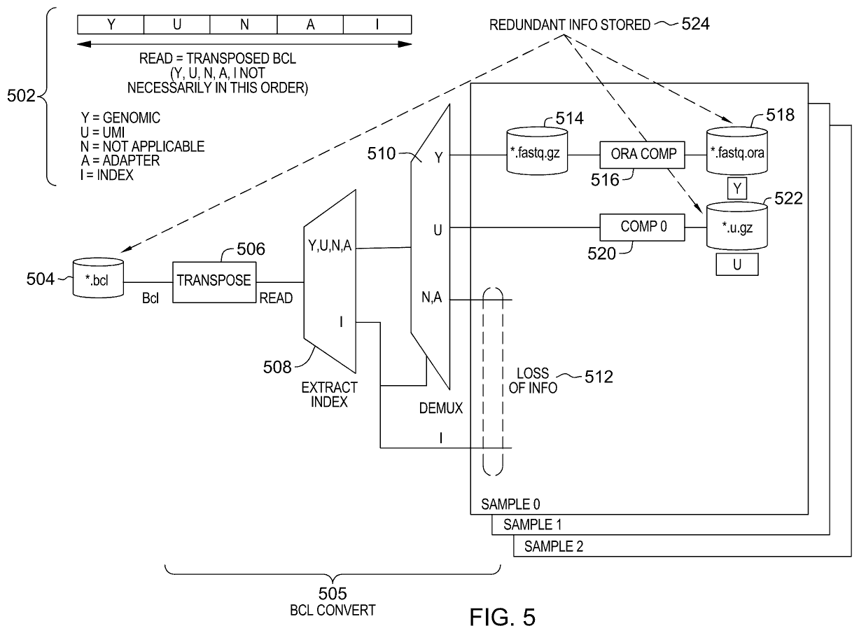

It is noted that the demultiplexing, transpose, and/or trim operations could be performed in a different order than shown in FIG. 1. In the example scenario of FIG. 1, there is an implied transposition of the index cycles, demux using the index cycles, storage of the demux information to disk (i.e., store which clusters are associated with a specific sample), and transposition (at 116) of the rest of the data. An example scenario in which the data is transposed and then demultiplexed to per-sample data after the transpose operation is depicted and described with reference to FIG. 5, below. As yet another scenario, the demultiplexing could occur after transposing the index cycles but with retention of demux information in working memory (i.e., to keep track of which clusters are associated with a specific sample) to enable further demuxing of the rest of the data.

Referring to FIG. 3, an example transpose operation takes per-cycle data as input and transposes the data into data for the whole read. Each column (302a, 302b, 302c) represents data for a respective single cycle collected across all clusters, which are represented by circles 304a, 304b, 304c, 304d, 304e. Thus, there are 5 clusters of data in this example collected across 3 cycles, which are transposed to data 306 for the whole read. Referring to FIG. 4, an example trim operation trims adapter information 402 from the genomic data 404 of interest, relying on the sample sheet to identify the adapters to be trimmed. Trimming causes a loss of information and is one reason the raw data may need to be retained.

The workflow continues by converting the FASTQ data to ORA data, i.e., by reading (11) the compressed FASTQ data from disk (106), decompressing (118) the FASTQ data and providing (12) it for compression (120) again into the ORA format (as ora.gz file(s)), which is/are written (13) again down to disk 106.

The workflow of FIG. 1 involves numerous intermediate files of differing formats and that are read/written to disk, which are relatively expensive input/output operations. Additionally, the resulting data is lossy in that at least some information of the raw (BCL) data that is critical to proper extraction of per-sample data from the raw data has been lost.

FIG. 5 depicts another example conventional workflow for sequence data formatting and compression. In this example, the transpose operation is performed prior to demultiplexing/trimming operations to produce the lossy, reformatted and compressed sequence data.

Referring to FIG. 5, read data 502 is provided in a transposed BCL arrangement of data of various types:
Y: genomic data of samples of interest;
U: Unique Molecular Identifiers (UMI) data, which is known information to guide secondary analysis in its processing;
N: Not-applicable (also called “not used”) data referring to data that are sequenced but are not needed for the particular application;
A: Adapter information of the adapters used during the sequencing run to attach to the genomic data of samples of interest to be combined with primers for amplification;
I: Index information (also referred to as barcode) for uniquely identifying per-sample data, i.e., which data is part of which sample of the sequence data

The Y data is genomic data of interest, while one or more of the U, N, A, and I data is example metadata. In this example of FIG. 5, the read (transposed BCL) data is presented in the order Y, U, N, A, I, though it could be provided in a different order. In addition, other metadata, or other types of data, of different types than those identified above may be present in the sequence data.

In FIG. 5, the workflow processing receives raw sequence data as BCL data 504 and inputs this to a sequence of BCL convert operations 505. Initially, a transpose operation 506 transposes the per-cycle data into whole reads, for instance reads with respective Y, U, N, A, and I data for each read. The read data is fed, on a per-read basis, into operation 508 (for instance a demultiplexing operation) that extracts index data (I) of the read. The extracted index data is used in a demultiplexing operation 510 to demultiplex read data into per-sample data of the read (Samples 0, 1, and 2 in this example). Specifically, the demultiplexing operation 510 demultiplexes, on a per-sample basis, the Y, U, N, and A data of the read. The I, N, and A data is lost 512. The per-sample genomic data Y is processed (for instance as described above) into compressed FASTQ data (FASTQ.gz file(s)) 514 and then ORA-compressed 516 into ORA-compressed data (FASTQ.ora file(s)) 518, in this example. Meanwhile, the U data is compressed 520 into compressed data (u.gz) 522 and retained. Conventionally, the relatively large BCL data 504 is maintained as is data 518 and 522, resulting in storage of redundant information (524).

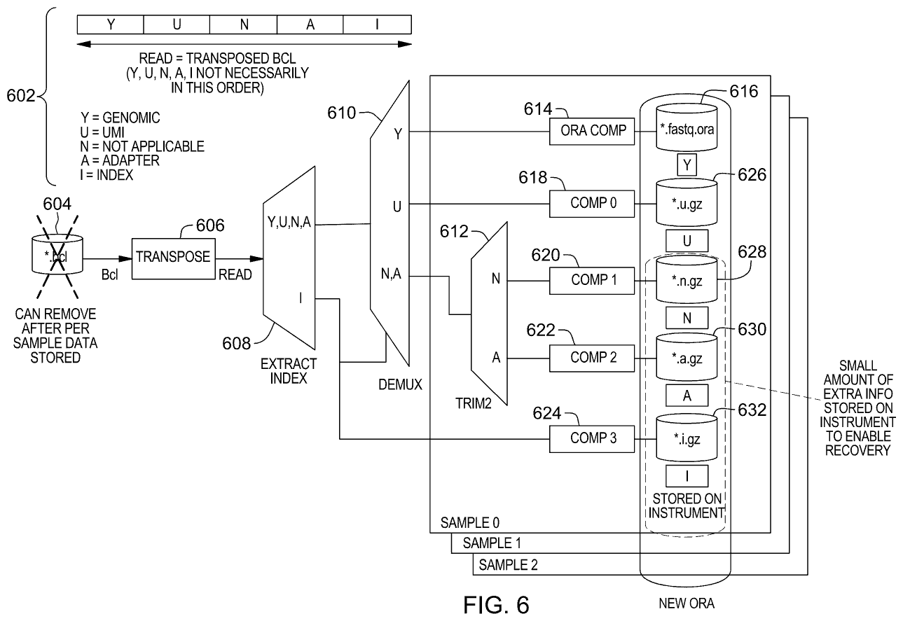

FIG. 6 depicts an example workflow for sequence data formatting and compression in accordance with aspects described herein. In this approach, separated per-sample genomic data, adapter data, and index data are stored to disk. In examples, the separated per-sample genomic data is stored in a format according to which the per-sample genomic data is compressed based on a reference sequence, for instance using ORA compression. In general, each of the different data types (e.g., Y, A, N, U, I) may be compressed using any respective, desired mechanism, approach, and/or format, for instance an approach/mechanism/format that is most efficient for that data type. In examples, the desired approach that may be most efficient for genomic data may be to utilize reference-based compression and store in a corresponding format.

Referring to FIG. 6, the read data 602 is provided in a transposed BCL arrangement of data of various types that include Y, U, N, A, and I data. The workflow receives this raw sequence data (e.g., as BCL data) 604 for the reads and inputs this to processing to ultimately provide per-sample genomic data for each sample. The processing includes a transpose operation 606 that transposes the input BCL data into whole reads, for instance reads with respective genomic data of interest (Y data) and metadata (U, N, A, and I data) for each read. The read data is fed, on a per-read basis, into operation 608 that extracts index data (I) of the read. The extracted index data is used in a demultiplexing operation 610 to demultiplex, using the index data, read data, for instance the remaining Y, U, N, A portions of the sequence data, into per-sample data of the read (Samples 0, 1, and 2 in this example). The demultiplexing operation 610 separates the genomic data of interest (Y) from other data, for instance the metadata. In this example, the Y data is separated from the U data and from the N and A data which are trimmed from the other data (Y and U data) and provided to trim operation 612 that separates the N data from the A data.

In some examples, the separating of the genomic data of interest from the metadata uses a configuration file that indicates the index associated with a sample, and separates the genomic data of interest from the metadata based on the indexes indicated by the configuration file.

Unlike in the conventional scenario of FIGS. 1 and 5, the I, N, and A data is not lost. Instead, in this example, the separated per-sample genomic data Y is processed (e.g., compressed) into compressed 614 ORA-compressed data (FASTQ.ora file(s)) 616, in this example. The Y data could have been compressed directly to ORA-compressed FASTQ data or could have been first converted to FASTQ format and then ORA-compressed from there, as was the case in FIG. 5.

The metadata may also be stored, optionally also in compressed format(s). Continuing with FIG. 6, compression operations 618, 620, 622, 624 compress the U data, N data, A data, and I data, respectively and separately in this example (though in others it may be possible to compress more than one data type together using the same compression), into compressed U data (u.gz) 626, N data (n.gz) 628, A data (a.gz) 630, and I data (i.gz) 632, respectively. In this manner, the separated per-sample genomic data (Y), the adapter data (A), and the index data (I), as well as the N and U data in this example, are all stored to disk, and in this example all in compressed format(s).

As a result of maintaining (at least) the A data and I data as 630, 632, the original sequence data (604) and intermediate data produced during the process can be removed/discarded. It is noted that any one or more of the U, N, A, and I data could be saved in the ORA file(s) themselves (that store the Y data), if desired, or in separate companion files. Thus, one or more files may be stored to disk/output, in which the separated per-sample genomic data Y (compressed in this example) could reside in the same file(s) or different data file(s) as the other sequence data (e.g. metadata, compressed in this example) being maintained.

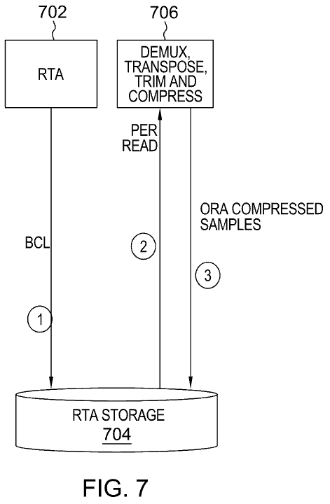

In some embodiments, the processing associated with the workflow of FIG. 6 is performed by the sequencing instrument itself that also sequenced the genomic material to produce and obtain the sequence data in the first place. The Y, U, N, A, and I data may be stored on the instrument to enable recovery of the sequencing data should the need arise. FIG. 7 depicts an example workflow implementing this, in which primary sequencing data analysis software 702 (RTA in this example) running on the sequencing instrument that sequences the genomic material produces sequence data as per-cycle BCL data that is initially written (1) to RTA storage 704. The BCL data is read (2) from RTA storage 704 (e.g., on a per-read basis), then processed 706 (demultiplexed, transposed, trimmed, and compressed) and written (3) to RTA storage as per-sample ORA-compressed data with the additional U, N, A, and I data as discussed above with reference to FIG. 6. In this example, the sequence data (BCLs) never leaves the instrument, and the BCLs can be deleted therefrom once the per-sample data has been stored at (3) with the additional data (e.g., at least the A and I data).

In this example, the read action at (2) can be performed only after a read's worth of data has been written out by RTA. Once the full read data has been provided in RTA storage 704, this can trigger action (2) and processing 706 of that read data to directly create the ORA-compressed data for each read sample.

In some alternative embodiments, the read data provided to RTA storage and read at (2) could already be demultiplexed (i.e., provided as per-sample) data, eliminating the need for demultiplexing processing as part of 706. That is, the demultiplexing action could be moved into RTA itself to use the indexes and output demultiplexed (per-sample) sequence data to the disk.

These aspects enable a workflow that eliminates the extra steps undertaken and disk storage used in the moving between various formats and the compression/decompression actions conventionally undertaken to convert from the sequence data to the per-sample ORA data. Thus, there is decreased resource (compute and storage) consumption and therefore decreased cost of processing and maintaining the data.

In the example of FIG. 7, the raw sequence data—BCL data in this example—does not need to leave the instrument, though it could be sent to an external device if desired. In any case, this raw sequence data is not needed to perform actions such as providing the data in FASTQ format. The data stored at (3) could be provided in the FASTQ format if desired, for instance using any known technique. A transparent FUSE layer can be provided and used to present the stored data as FASTQ data directly, if desired, as opposed to first decompressing the data for presentation in FASTQ format. In this regard, ORA files can be presented as FASTQ data, for instance by pre-loading a library for any tool that expects FASTQ data as input and loading the ORA data via the library, or alternatively by mounting the ORA data directory with a transparent layer that presents the data as if it were provided from FASTQ file(s). This provides an alternative to a discrete conversion approach, for instance, that converts the ORA data to FASTQ data and stores/presents the FASTQ data as such.

Advantageously, aspects can provide per-sample genomic data on which secondary analysis could be performed directly, and this could be provided directly from a sequencer. The per-sample data can be provided losslessly on account of the additional data, for instance additional adapter, index, and N data—useful information from the raw sequencing data that enables the raw sequencing data to be reconstructed, if desired, and potentially reprocessed (demux, trim, transpose) to produce-again the per-sample genomic data, if desired, for instance in the event of an error such as an erroneous or missing sample sheet. The additional information being retained provides the information to correct any mistakes made with the sample sheet or if it's missing, for instance. Thus, it is possible to reconstruct the raw sequence data (BCLs) from the ORA-compressed and additional data, if desired, using the retained additional data. This might be done in situations where it is desired to re-demultiplex the sequence data, for instance if there were errors made in the initial demultiplexing processing.

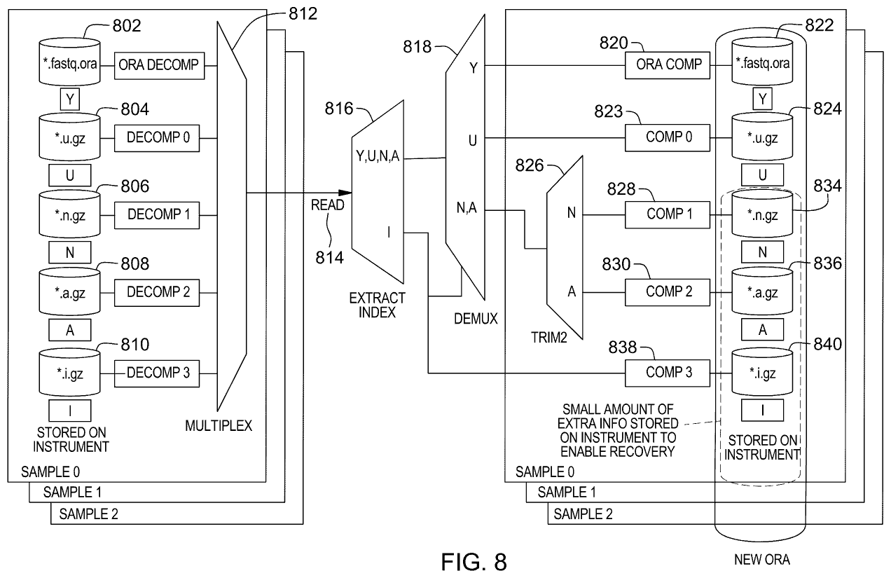

FIG. 8 depicts such an example of a recovery scenario in accordance with aspects described herein. The recovery can be initiated based on a request, for instance one that requests to recover sequence data from stored, compressed genomic data and metadata. In FIG. 8, the recovery recovers the read data of the reads, including the per-sample genomic data for one or more samples of each such read, from the stored data of FIG. 6, i.e., the compressed per-sample genomic data (Y) 802 for the samples together with the compressed U data 804, compressed N data 806, compressed A data 808, and compressed I data 810 of those samples. A process reads and decompresses this data, for instance via ORA-based decompression of the per-sample genomic data 802 and whichever decompression mechanism(s) correlate to the compression applied to the U, N, A, and I data. The decompression provides decompressed genomic data of interest (i.e., the Y data, the genomic data of interested that was previously separated from the metadata as described above). The decompressed genomic data of interest and the other data (which was also decompressed in this example) is then combined, for instance is multiplexed 812. This rebuilds the full read data 814, including Y, U, N, A, and I data for each read, as the sequence data that had been previously provided in FIG. 6.

The read data 814 can be provided for any desired use. In one example, as in FIG. 8, it may be reprocessed in which the index information is extracted 816 and used to demultiplex 818 the Y, U, and N and A data for compression (820, 823, respectively) of the Y and U data and storage of that compressed data as 822, 824, as well as separate trimming 826 of the N data from the A data and compression of both (828, 830) to produce compressed N and A data 834, 836. Additionally, the I data is compressed 838 and stored 840.

In some embodiments, the index and adapter data that is stored could later be discarded if the demultiplexing has been confirmed to be correct. In these situations, the raw sequence data could not be recovered as discussed above, however this may not be necessary if it is determined that the per-sample data (from the demultiplexing action) has been determined to be correct.

In some embodiments, the genomic data of interest is not isolated from some/all of the metadata. For instance, a process could obtain sequence data that includes genomic data of interest and metadata, and process this by compressing the genomic data of interest and metadata to produce compressed data which the process then stores, for instance to disk. The compression could be performed based on a reference sequence, for instance by using a reference-based compression technique, such as ORA compression as one example. At some point based on a request to recover the sequence data from the stored compresses genomic data and metadata that was stored (to disk, for instance), a process could perform the recovery by decompressing the compressed data to provide decompressed genomic data of interest and metadata, i.e., the sequence data that was previously obtained and compressed.

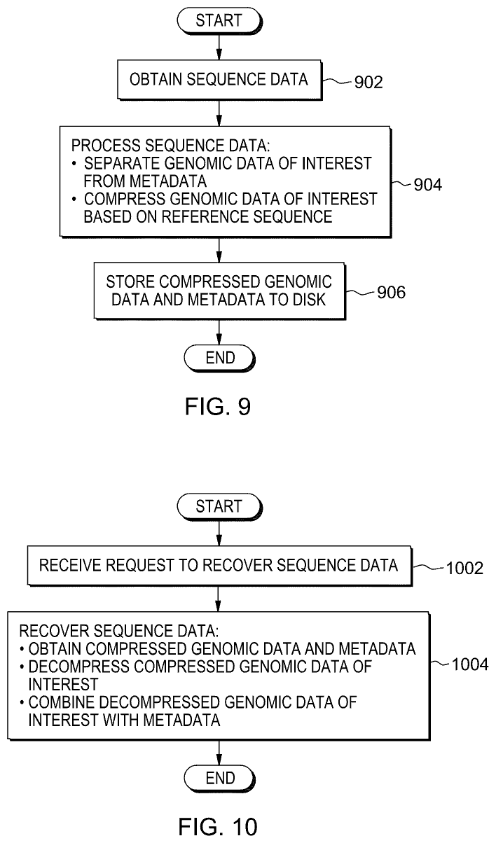

FIGS. 9 and 10 depict example processes for sequence data processing, retention, and recovery. The processes may be executed, in one or more examples, by a processor or processing circuitry, for instance that of one or more computers/computer systems. A computer system could be of, or in communication with, a sequencing/sequencer device, for example. Some or all aspects of the process of FIG. 9 could be performed by a different one or more system(s) than some or all aspects of the process of FIG. 10.

FIG. 9 depicts an example process for sequence data processing and retention, in accordance with aspects described herein. The process of FIG. 9 includes obtaining (902) sequence data, for instance sequence data produced by a sequencer device. The sequence data includes genomic data of interest and metadata. The genomic data of interest includes, as examples, base calls or nucleotide bases. Example such metadata can include, but is not limited to, adapter data, N data, index data, and UMI data. Example adapter data is, or includes, adapter information of adapters used during a sequencing run to attach to genomic data of samples of interest to be combined with primers for amplification. Example N data is, or includes, data that is sequenced but not needed for a particular application. Example index data is, or includes, index or barcode information for uniquely identifying per-sample data. Example UMI data is known information to guide secondary analysis processing.

The process of FIG. 9 continues by processing (904) the sequence data. The processing includes, in this example, separating the genomic data of interest from the metadata and compressing the separated genomic data of interest based on a reference sequence to produce compressed genomic data. In embodiments, the separating uses a configuration file that indicates indexes, and the separating separates the genomic data of interest from the metadata based on the indexes indicated by the configuration file.

The processing can trim at least some of the metadata from other data of the sequence data. For instance, the separating can trim the genomic data of interest from at least some of the metadata, for example adapter, UMI, N, and/or index data, as examples.

FIG. 9 continues by storing (906) the compressed genomic data and the metadata, for instance storing it to disk.

There are situations in which processing the sequence data does not separate the genomic data of interest from the metadata. For instance, there could be situations where index data is provided but it is not known as such to the process and no separating/demultiplexing action is taken against the sequence data to separate the genomic data of interest from other data of the sequence data. Thus, in an example process in accordance with aspects described herein, the process obtains sequence data produced by a sequencer device and including genomic data of interest and metadata, processes the sequence data, which includes compressing the genomic data of interest and metadata based on a reference sequence to produce compressed data, and stores the compressed data, for instance stores it to disk.

In embodiments where separation is provided, the separating can include using the index data to demultiplex at least a portion of the sequence data (for instance the Y, U, N, and A data) to provide the separated genomic data of interest (Y data) as per-sample genomic data. The compressing can thereby provide compressed per-sample genomic data as the compressed genomic data. Meanwhile, the storing can store the index data, for instance to the disk.

As noted, the trimmed metadata can include adapter data, UMI data, and/or data selected to be ignored (N data), as examples. The storing can therefore store the adapter data, UMI data, and/or data selected to be ignored, for instance to the disk. The processing can further includes compressing the metadata to provide compressed metadata, and therefore the storing can store this compressed metadata, for instance to the disk.

The compressed genomic data and the metadata (compressed or not) can be stored in the same or different file(s). In some examples, the storing stores the compressed genomic data, for instance to the disk, in a first one or more data files and stores the metadata, for instance to the disk, in a second one or more data files different from the first one or more data files. Alternatively, the storing stores the compressed genomic data in one or more data files that also store the metadata.

Some or all aspects discussed above could be performed by a sequencer device (“instrument”), if desired, using BCL data generated by the instrument. In embodiments, the process of FIG. 9 further includes performing a sequencing run by a sequence device. The sequencing sequences genomic material to produce and obtain the sequence data. Further, the sequencer device could perform the obtaining, the processing, and the storing of FIG. 9, where the disk (to which the compressed genomic data and metadata are stored in embodiments) is a storage device of the sequencer device.

Additional aspects described herein provide for recovery of sequence data. FIG. 10 depicts an example process for sequence data recovery, in accordance with aspects described herein. The process of FIG. 10 includes receiving (1002) a request to recover sequence data, for instance to recover sequence data from stored compressed genomic data and metadata. As an example, the request could be a request to recover sequence data that was processed and retained as discussed above with reference to FIG. 9. Based on this request, the process recovers (1004) the sequence data from the stored compressed genomic data and metadata stored, for instance to disk. In this example, the recovering includes obtaining stored compressed genomic data and the metadata. For instance, per the process of FIG. 9, the compressed genomic data could include genomic data of interest that was compressed based on a reference sequence. The recovery 1004 also includes decompressing the compressed genomic data to provide decompressed genomic data of interest. The produced decompressed genomic data of interest is, for instance, the separated genomic data of interest discussed with reference to FIG. 9. The recovery 1004 further includes combining the decompressed genomic data of interest with the metadata to provide the sequence data requested to be recovered.

In examples, the sequence data includes data for a plurality of reads, the metadata includes index data for the plurality of reads, and the combining includes remultiplexing the decompressed genomic data of interest with the metadata.

The metadata could itself have been compressed and therefore the recovery 1004 could include decompressing the compressed metadata to provide decompressed metadata as the metadata that is combined with the decompressed genomic data of interest to provide the sequence data.

The sequence data provided by the process of FIG. 10 could be provided for any desired use. One such use is sequence data processing in accordance with the previously described FIG. 9. Thus, in examples, aspects of FIG. 9 may be performed after recovering the data (FIG. 10, #1004), and more specifically the sequence data provided at 1004 could be processed (#904 of FIG. 9) to separate and compress genomic data of interest, and (#906 of FIG. 9) store the compressed genomic data and metadata, for instance to disk.

As noted, there are situations in which the genomic data and metadata may not have been separated. Thus, in an example process in accordance with aspects described herein, based on a request to recover sequence data from stored compressed genomic data and metadata, the process obtains the compressed genomic data and metadata, the compressed genomic data and metadata including genomic data of interest compressed based on a reference sequence, and decompressing the compressed genomic data and metadata to provide decompressed genomic data of interest and metadata as the sequence data requested to be recovered.

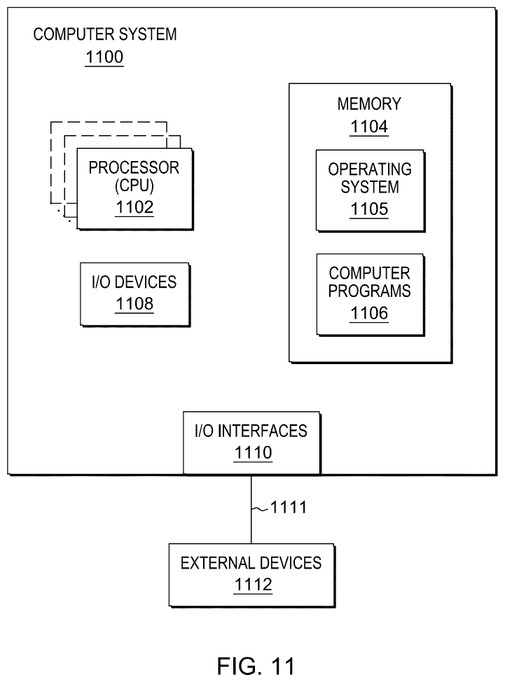

Processes described herein may be performed singly or collectively by one or more computer systems, such as one or more computer systems of, or in communication with, a sequencing/sequencer device, or any other computer system(s), as examples. FIG. 11 depicts one example of such a computer system and associated devices to incorporate and/or use aspects described herein. A computer system may also be referred to herein as a data processing device/system, computing device/system/node, or simply a computer. The computer system may be based on one or more of various system architectures and/or instruction set architectures.

FIG. 11 shows a computer system 1100 in communication with external device(s) 1112. Computer system 1100 includes one or more processor(s) 1102, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 1102 can also include register(s) to be used by one or more of the functional components. Computer system 1100 also includes memory 1104, input/output (I/O) devices 1108, and I/O interfaces 1110, which may be coupled to processor(s) 1102 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 1104 can be or include main or system memory (e.g. Random Access Memory) used in the execution of program instructions, storage device(s) such as hard drive(s), flash media, or optical media as examples, and/or cache memory, as examples. Memory 1104 can include, for instance, a cache, such as a shared cache, which may be coupled to local caches (examples include L1 cache, L2 cache, etc.) of processor(s) 1102. Additionally, memory 1104 may be or include at least one computer program product having a set (e.g., at least one) of program modules, instructions, code or the like that is/are configured to carry out functions of embodiments described herein when executed by one or more processors.

Memory 1104 can store an operating system 1105 and other computer programs 1106, such as one or more computer programs/applications that execute to perform aspects described herein. Specifically, programs/applications can include computer readable program instructions that may be configured to carry out functions of embodiments of aspects described herein.

Examples of I/O devices 1108 include but are not limited to microphones, speakers, Global Positioning System (GPS) devices, cameras, lights, accelerometers, gyroscopes, magnetometers, sensor devices configured to sense light, proximity, heart rate, body and/or ambient temperature, blood pressure, and/or skin resistance, and activity monitors. An I/O device may be incorporated into the computer system as shown, though in some embodiments an I/O device may be regarded as an external device (1112) coupled to the computer system through one or more I/O interfaces 1110.

Computer system 1100 may communicate with one or more external devices 1112 via one or more I/O interfaces 1110. Example external devices include a keyboard, a pointing device, a display, a sequencing instrument, and/or any other devices that enable a user to interact with computer system 1100. Other example external devices include any device that enables computer system 1100 to communicate with one or more other computing systems or peripheral devices such as a printer. A network interface/adapter is an example I/O interface that enables computer system 1100 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 1110 and external devices 1112 can occur across wired and/or wireless communications link(s) 1111, such as Ethernet-based wired or wireless connections. Example wireless connections include cellular, Wi-Fi, Bluetooth®, proximity-based, near-field, or other types of wireless connections. More generally, communications link(s) 1111 may be any appropriate wireless and/or wired communication link(s) for communicating data.

Particular external device(s) 1112 may include one or more data storage devices, which may store one or more programs, one or more computer readable program instructions, and/or data, etc. Computer system 1100 may include and/or be coupled to and in communication with (e.g. as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 1100 may be operational with numerous other general purpose or special purpose computing system environments or configurations. Computer system 1100 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Aspects of the present invention may be a system, a method, and/or a computer program product, any of which may be configured to perform or facilitate aspects described herein.

In some embodiments, aspects may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g. instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Although various embodiments are described above, these are only examples.

Provided is a small sampling of embodiments of the present disclosure, as described herein:

A1. A computer-implemented method comprising: obtaining sequence data produced by a sequencer device, the sequence data comprising genomic data of interest and metadata; processing the sequence data, the processing comprising: separating the genomic data of interest from the metadata; and compressing the separated genomic data of interest based on a reference sequence to produce compressed genomic data; and storing the compressed genomic data and the metadata.

A2. The method of A1, wherein the separating uses a configuration file that indicates indexes, and the separating separates the genomic data of interest from the metadata based on the indexes indicated by the configuration file.

A3. The method of A1, wherein the metadata comprises index data for a plurality of reads.

A4. The method of A3, wherein the separating comprises using the index data to demultiplex at least a portion of the sequence data to provide the separated genomic data of interest as per-sample genomic data, wherein the compressing provides compressed per-sample genomic data as the compressed genomic data, and wherein the storing stores the index data.

A5. The method of A1, wherein the processing trims at least some of the metadata from other data of the sequence data.

A6. The method of A5, wherein the trimmed metadata comprises adapter data, Unique Molecular Identifiers (UMI) data, and/or data selected to be ignored, and wherein the storing stores each of the adapter data, Unique Molecular Identifiers (UMI) data, and/or data selected to be ignored.

A7. The method of A1, wherein the processing further comprises compressing the metadata to provide compressed metadata, wherein the storing stores the compressed metadata.

A8. The method of A1, wherein the storing stores the compressed genomic data in a first one or more data files and stores the metadata in a second one or more data files different from the first one or more data files.

A9. The method of A1, wherein the storing stores the compressed genomic data in one or more data files that also store the metadata.

A10. The method of A1, further comprising, based on a request, recovering the sequence data from the stored compressed genomic data and metadata, the recovering comprising: decompressing the compressed genomic data to provide decompressed genomic data of interest as the separated genomic data of interest; and combining the decompressed genomic data of interest with the metadata to provide combined genomic data and metadata.

A11. The method of A10, wherein the metadata comprises index data for a plurality of reads, wherein the separating comprises using the index data to demultiplex at least a portion of the sequence data to provide the separated genomic data of interest as per-sample genomic data, wherein the compressing provides compressed per-sample genomic data as the compressed genomic data, wherein the storing stores the index data, and wherein the combining comprises remultiplexing the decompressed genomic data of interest with the metadata to provide the combined genomic data and metadata.

A12. The method of A10, wherein the processing further comprises compressing the metadata to provide compressed metadata, wherein the storing stores the compressed metadata, and wherein the recovering further comprises decompressing the compressed metadata to provide decompressed metadata as the metadata that is combined with the decompressed genomic data of interest.

A13. The method of A1, further comprising sequencing, by the sequencer device, genomic material to produce and obtain the sequence data, wherein the sequencer device performs the obtaining, the processing, and the storing, and wherein the storing stores the compressed genomic data and the metadata to a storage device of the sequencer device.

A14. A computer system comprising: a memory; and a processor in communication with the memory, wherein the computer system is configured to perform a method that includes obtaining sequence data produced by a sequencer device, the sequence data comprising genomic data of interest and metadata; processing the sequence data, the processing comprising: separating the genomic data of interest from the metadata; and compressing the separated genomic data of interest based on a reference sequence to produce compressed genomic data; and storing the compressed genomic data and the metadata.

A15. The computer system of A14, wherein the separating uses a configuration file that indicates indexes, and the separating separates the genomic data of interest from the metadata based on the indexes indicated by the configuration file.

A16. The computer system of A14, wherein the metadata comprises index data for a plurality of reads.

A17. The computer system of A16, wherein the separating comprises using the index data to demultiplex at least a portion of the sequence data to provide the separated genomic data of interest as per-sample genomic data, wherein the compressing provides compressed per-sample genomic data as the compressed genomic data, and wherein the storing stores the index data.

A18. The computer system of A14, wherein the processing trims at least some of the metadata from other data of the sequence data.

A19. The computer system of A18, wherein the trimmed metadata comprises adapter data, Unique Molecular Identifiers (UMI) data, and/or data selected to be ignored, and wherein the storing stores each of the adapter data, Unique Molecular Identifiers (UMI) data, and/or data selected to be ignored.

A20. The computer system of A14, wherein the processing further comprises compressing the metadata to provide compressed metadata, wherein the storing stores the compressed metadata.

A21. The computer system of A14, wherein the storing stores the compressed genomic data in a first one or more data files and stores the metadata in a second one or more data files different from the first one or more data files.

A22. The computer system of A14, wherein the storing stores the compressed genomic data in one or more data files that also store the metadata.

A23. The computer system of A14, wherein the method further includes, based on a request, recovering the sequence data from the stored compressed genomic data and metadata, the recovering comprising: decompressing the compressed genomic data to provide decompressed genomic data of interest as the separated genomic data of interest; and combining the decompressed genomic data of interest with the metadata to provide combined genomic data and metadata.

A24. The computer system of A23, wherein the metadata comprises index data for a plurality of reads, wherein the separating comprises using the index data to demultiplex at least a portion of the sequence data to provide the separated genomic data of interest as per-sample genomic data, wherein the compressing provides compressed per-sample genomic data as the compressed genomic data, wherein the storing stores the index data, and wherein the combining comprises remultiplexing the decompressed genomic data of interest with the metadata to provide the combined genomic data and metadata.

A25. The computer system of A23, wherein the processing further comprises compressing the metadata to provide compressed metadata, wherein the storing stores the compressed metadata, and wherein the recovering further comprises decompressing the compressed metadata to provide decompressed metadata as the metadata that is combined with the decompressed genomic data of interest.

A26. The computer system of A14, wherein the method further includes sequencing, by the sequencer device, genomic material to produce and obtain the sequence data, wherein the sequencer device performs the obtaining, the processing, and the storing, and wherein the storing stores the compressed genomic data and the metadata to a storage device of the sequencer device.

A27. A computer program product comprising: a computer readable storage medium readable by a processing circuit and storing instructions for execution by the processing circuit to perform a method that includes obtaining sequence data produced by a sequencer device, the sequence data comprising genomic data of interest and metadata; processing the sequence data, the processing comprising: separating the genomic data of interest from the metadata; and compressing the separated genomic data of interest based on a reference sequence to produce compressed genomic data; and storing the compressed genomic data and the metadata.

A28. The computer program product of A27, wherein the separating uses a configuration file that indicates indexes, and the separating separates the genomic data of interest from the metadata based on the indexes indicated by the configuration file.

A29. The computer program product of A27, wherein the metadata comprises index data for a plurality of reads.

A30. The computer program product of A29, wherein the separating comprises using the index data to demultiplex at least a portion of the sequence data to provide the separated genomic data of interest as per-sample genomic data, wherein the compressing provides compressed per-sample genomic data as the compressed genomic data, and wherein the storing stores the index data.

A31. The computer program product of A27, wherein the processing trims at least some of the metadata from other data of the sequence data.

A32. The computer program product of A31, wherein the trimmed metadata comprises adapter data, Unique Molecular Identifiers (UMI) data, and/or data selected to be ignored, and wherein the storing stores each of the adapter data, Unique Molecular Identifiers (UMI) data, and/or data selected to be ignored.

A33. The computer program product of A27, wherein the processing further comprises compressing the metadata to provide compressed metadata, wherein the storing stores the compressed metadata.

A34. The computer program product of A27, wherein the storing stores the compressed genomic data in a first one or more data files and stores the metadata in a second one or more data files different from the first one or more data files.

A35. The computer program product of A27, wherein the storing stores the compressed genomic data in one or more data files that also store the metadata.

A36. The computer program product of A27, wherein the method further includes, based on a request, recovering the sequence data from the stored compressed genomic data and metadata, the recovering comprising: decompressing the compressed genomic data to provide decompressed genomic data of interest as the separated genomic data of interest; and combining the decompressed genomic data of interest with the metadata to provide combined genomic data and metadata.

A37. The computer program product of A36, wherein the metadata comprises index data for a plurality of reads, wherein the separating comprises using the index data to demultiplex at least a portion of the sequence data to provide the separated genomic data of interest as per-sample genomic data, wherein the compressing provides compressed per-sample genomic data as the compressed genomic data, wherein the storing stores the index data, and wherein the combining comprises remultiplexing the decompressed genomic data of interest with the metadata to provide the combined genomic data and metadata.

A38. The computer program product of A36, wherein the processing further comprises compressing the metadata to provide compressed metadata, wherein the storing stores the compressed metadata, and wherein the recovering further comprises decompressing the compressed metadata to provide decompressed metadata as the metadata that is combined with the decompressed genomic data of interest.

A39. The computer program product of A27, wherein the method further includes sequencing, by the sequencer device, genomic material to produce and obtain the sequence data, wherein the sequencer device performs the obtaining, the processing, and the storing, and wherein the storing stores the compressed genomic data and the metadata to a storage device of the sequencer device.

B1. A computer-implemented method comprising: based on a request to recover sequence data from stored compressed genomic data and metadata: obtaining the stored compressed genomic data and the metadata, the compressed genomic data comprising genomic data of interest compressed based on a reference sequence; decompressing the compressed genomic data to provide decompressed genomic data of interest; and combining the decompressed genomic data of interest with the metadata to provide the sequence data.

B2. The method of B1, wherein the sequence data comprises data for a plurality of reads, wherein the metadata comprises index data for the plurality of reads, and wherein the combining comprises remultiplexing the decompressed genomic data of interest with the metadata.

B3. A computer system comprising: a memory; and a processor in communication with the memory, wherein the computer system is configured to perform a method that includes: based on a request to recover sequence data from stored compressed genomic data and metadata: obtaining the stored compressed genomic data and the metadata, the compressed genomic data comprising genomic data of interest compressed based on a reference sequence; decompressing the compressed genomic data to provide decompressed genomic data of interest; and combining the decompressed genomic data of interest with the metadata to provide the sequence data.

B4. The computer system method of B3, wherein the sequence data comprises data for a plurality of reads, wherein the metadata comprises index data for the plurality of reads, and wherein the combining comprises remultiplexing the decompressed genomic data of interest with the metadata.

B5. A computer program product comprising: a computer readable storage medium readable by a processing circuit and storing instructions for execution by the processing circuit to perform a method that includes: based on a request to recover sequence data from stored compressed genomic data and metadata: obtaining the stored compressed genomic data and the metadata, the compressed genomic data comprising genomic data of interest compressed based on a reference sequence; decompressing the compressed genomic data to provide decompressed genomic data of interest; and combining the decompressed genomic data of interest with the metadata to provide the sequence data.

B6. The computer program product of B5, wherein the sequence data comprises data for a plurality of reads, wherein the metadata comprises index data for the plurality of reads, and wherein the combining comprises remultiplexing the decompressed genomic data of interest with the metadata.

C1. A computer-implemented method comprising: obtaining sequence data produced by a sequencer device, the sequence data comprising genomic data of interest and metadata; processing the sequence data, the processing comprising compressing the genomic data of interest and metadata based on a reference sequence to produce compressed data; and storing the compressed data.

C2. The method of C1, further comprising, based on a request, recovering the sequence data from the stored compressed data, the recovering comprising decompressing the compressed data to provide decompressed genomic data of interest and metadata.

C3. A computer system comprising: a memory; and a processor in communication with the memory, wherein the computer system is configured to perform a method that includes: obtaining sequence data produced by a sequencer device, the sequence data comprising genomic data of interest and metadata; processing the sequence data, the processing comprising compressing the genomic data of interest and metadata based on a reference sequence to produce compressed data; and storing the compressed data.

C4. The computer system of C3, further comprising, based on a request, recovering the sequence data from the stored compressed data, the recovering comprising decompressing the compressed data to provide decompressed genomic data of interest and metadata.

C5. A computer program product comprising: a computer readable storage medium readable by a processing circuit and storing instructions for execution by the processing circuit to perform a method that includes: obtaining sequence data produced by a sequencer device, the sequence data comprising genomic data of interest and metadata; processing the sequence data, the processing comprising compressing the genomic data of interest and metadata based on a reference sequence to produce compressed data; and storing the compressed data.

C6. The computer program product of C5, further comprising, based on a request, recovering the sequence data from the stored compressed data, the recovering comprising decompressing the compressed data to provide decompressed genomic data of interest and metadata

D1. A computer-implemented method comprising: based on a request to recover sequence data from stored compressed genomic data and metadata: obtaining the compressed genomic data and metadata, the compressed genomic data and metadata comprising genomic data of interest compressed based on a reference sequence; and decompressing the compressed genomic data and metadata to provide decompressed genomic data of interest and metadata as the sequence data.

D2. A computer system comprising: a memory; and a processor in communication with the memory, wherein the computer system is configured to perform a method that includes: based on a request to recover sequence data from stored compressed genomic data and metadata: obtaining the compressed genomic data and metadata, the compressed genomic data and metadata comprising genomic data of interest compressed based on a reference sequence; and decompressing the compressed genomic data and metadata to provide decompressed genomic data of interest and metadata as the sequence data.

D3. A computer program product comprising: a computer readable storage medium readable by a processing circuit and storing instructions for execution by the processing circuit to perform a method that includes: based on a request to recover sequence data from stored compressed genomic data and metadata: obtaining the compressed genomic data and metadata, the compressed genomic data and metadata comprising genomic data of interest compressed based on a reference sequence; and decompressing the compressed genomic data and metadata to provide decompressed genomic data of interest and metadata as the sequence data.

The terminology used herein is for the purpose of describing particular embodiments only and is not intended to be limiting. As used herein, the singular forms “a” “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method comprising:
obtaining sequence data produced by a sequencer device, the sequence data comprising genomic data of interest and metadata;
processing the sequence data, the processing comprising:
separating the genomic data of interest from the metadata; and
compressing the separated genomic data of interest based on a reference sequence to produce compressed genomic data; and
storing the compressed genomic data and the metadata.

2. The method of claim 1, wherein the separating uses a configuration file that indicates indexes, and the separating separates the genomic data of interest from the metadata based on the indexes indicated by the configuration file.

3. The method of claim 1, wherein the metadata comprises index data for a plurality of reads.

4. The method of claim 3, wherein the separating comprises using the index data to demultiplex at least a portion of the sequence data to provide the separated genomic data of interest as per-sample genomic data, wherein the compressing provides compressed per-sample genomic data as the compressed genomic data, and wherein the storing stores the index data.

5. The method of claim 1, wherein the processing trims at least some of the metadata from other data of the sequence data.

6. The method of claim 5, wherein the trimmed metadata comprises adapter data, Unique Molecular Identifiers (UMI) data, and/or data selected to be ignored, and wherein the storing stores each of the adapter data, Unique Molecular Identifiers (UMI) data, and/or data selected to be ignored.

7. The method of claim 1, wherein the processing further comprises compressing the metadata to provide compressed metadata, wherein the storing stores the compressed metadata.

8. The method of claim 1, wherein the storing stores the compressed genomic data in a first one or more data files and stores the metadata in a second one or more data files different from the first one or more data files.

9. The method of claim 1, wherein the storing stores the compressed genomic data in one or more data files that also store the metadata.

10. The method of claim 1, further comprising, based on a request, recovering the sequence data from the stored compressed genomic data and metadata, the recovering comprising:
decompressing the compressed genomic data to provide decompressed genomic data of interest as the separated genomic data of interest; and
combining the decompressed genomic data of interest with the metadata to provide combined genomic data and metadata.

11. The method of claim 10, wherein the metadata comprises index data for a plurality of reads, wherein the separating comprises using the index data to demultiplex at least a portion of the sequence data to provide the separated genomic data of interest as per-sample genomic data, wherein the compressing provides compressed per-sample genomic data as the compressed genomic data, wherein the storing stores the index data, and wherein the combining comprises remultiplexing the decompressed genomic data of interest with the metadata to provide the combined genomic data and metadata.

12. The method of claim 10, wherein the processing further comprises compressing the metadata to provide compressed metadata, wherein the storing stores the compressed metadata, and wherein the recovering further comprises decompressing the compressed metadata to provide decompressed metadata as the metadata that is combined with the decompressed genomic data of interest.

13. The method of claim 1, further comprising sequencing, by the sequencer device, genomic material to produce and obtain the sequence data, wherein the sequencer device performs the obtaining, the processing, and the storing, and wherein the storing stores the compressed genomic data and the metadata to a storage device of the sequencer device.

14. A computer system comprising:
a memory; and
a processor in communication with the memory, wherein the computer system is configured to perform a method that includes:
obtaining sequence data produced by a sequencer device, the sequence data comprising genomic data of interest and metadata;
processing the sequence data, the processing comprising:
separating the genomic data of interest from the metadata; and
compressing the separated genomic data of interest based on a reference sequence to produce compressed genomic data; and
storing the compressed genomic data and the metadata.

15. The computer system of claim 14, wherein the separating uses a configuration file that indicates indexes, and the separating separates the genomic data of interest from the metadata based on the indexes indicated by the configuration file.

16. The computer system of claim 14, wherein the metadata comprises index data for a plurality of reads, wherein the separating comprises using the index data to demultiplex at least a portion of the sequence data to provide the separated genomic data of interest as per-sample genomic data, wherein the compressing provides compressed per-sample genomic data as the compressed genomic data, and wherein the storing stores the index data.

17. The computer system of claim 14, wherein the processing trims at least some of the metadata from other data of the sequence data, wherein the trimmed metadata comprises adapter data, Unique Molecular Identifiers (UMI) data, and/or data selected to be ignored, and wherein the storing stores each of the adapter data, Unique Molecular Identifiers (UMI) data, and/or data selected to be ignored.

18. The computer system of claim 14, wherein the method further comprises, based on a request, recovering the sequence data from the stored compressed genomic data and metadata, the recovering comprising:
decompressing the compressed genomic data to provide decompressed genomic data of interest as the separated genomic data of interest; and
combining the decompressed genomic data of interest with the metadata to provide combined genomic data and metadata.

19. The computer system of claim 18, wherein the metadata comprises index data for a plurality of reads, wherein the separating comprises using the index data to demultiplex at least a portion of the sequence data to provide the separated genomic data of interest as per-sample genomic data, wherein the compressing provides compressed per-sample genomic data as the compressed genomic data, wherein the storing stores the index data, and wherein the combining comprises remultiplexing the decompressed genomic data of interest with the metadata to provide the combined genomic data and metadata.

20. The computer system of claim 18, wherein the processing further comprises compressing the metadata to provide compressed metadata, wherein the storing stores the compressed metadata, and wherein the recovering further comprises decompressing the compressed metadata to provide decompressed metadata as the metadata that is combined with the decompressed genomic data of interest.

21. A computer program product comprising:
a computer readable storage medium readable by a processing circuit and storing instructions for execution by the processing circuit to perform a method that includes:
obtaining sequence data produced by a sequencer device, the sequence data comprising genomic data of interest and metadata;
processing the sequence data, the processing comprising:
separating the genomic data of interest from the metadata; and
compressing the separated genomic data of interest based on a reference sequence to produce compressed genomic data; and
storing the compressed genomic data and the metadata.

22. The computer program product of claim 21, wherein the separating uses a configuration file that indicates indexes, and the separating separates the genomic data of interest from the metadata based on the indexes indicated by the configuration file.

23. The computer program product of claim 21, wherein the metadata comprises index data for a plurality of reads, wherein the separating comprises using the index data to demultiplex at least a portion of the sequence data to provide the separated genomic data of interest as per-sample genomic data, wherein the compressing provides compressed per-sample genomic data as the compressed genomic data, and wherein the storing stores the index data.

24. The computer program product of claim 21, wherein the processing trims at least some of the metadata from other data of the sequence data, wherein the trimmed metadata comprises adapter data, Unique Molecular Identifiers (UMI) data, and/or data selected to be ignored, and wherein the storing stores each of the adapter data, Unique Molecular Identifiers (UMI) data, and/or data selected to be ignored.

25. The computer program product of claim 21, wherein the method further comprises, based on a request, recovering the sequence data from the stored compressed genomic data and metadata, the recovering comprising:
decompressing the compressed genomic data to provide decompressed genomic data of interest as the separated genomic data of interest; and
combining the decompressed genomic data of interest with the metadata to provide combined genomic data and metadata.

26. The computer program product of claim 25, wherein the metadata comprises index data for a plurality of reads, wherein the separating comprises using the index data to demultiplex at least a portion of the sequence data to provide the separated genomic data of interest as per-sample genomic data, wherein the compressing provides compressed per-sample genomic data as the compressed genomic data, wherein the storing stores the index data, and wherein the combining comprises remultiplexing the decompressed genomic data of interest with the metadata to provide the combined genomic data and metadata.

27. The computer program product of claim 25, wherein the processing further comprises compressing the metadata to provide compressed metadata, wherein the storing stores the compressed metadata, and wherein the recovering further comprises decompressing the compressed metadata to provide decompressed metadata as the metadata that is combined with the decompressed genomic data of interest.
